(19) United States
(12) Patent Application Publication
(10) Pub. No.: US 2004/0115620 A1
(43) Pub. Date: Jun. 17, 2004

(54) Adenoviral replicons

(76) Inventors: Menzo Havenga, KG Alphen a/d Rijn (NL); Ronald H P Brus, Voorschoten (NL)

Correspondence Address: Allen C Turner, TraskBritt, P O Box 2550, Salt Lake City, UT, 84110, US

(21) Appl. No.: 10/432,105

(22) PCT Filed: Nov. 19, 2001
(86) PCT No.: PCT/NL01/00834

(30) Foreign Application Priority Data
Nov. 20, 2000 (EP) 00204097.0

(51) Int. Cl. C12Q 1/70

(52) U.S. Cl. 435/5

(57) ABSTRACT

The invention provides a method for identifying an adenoviral replicon capable of eliminating a target cell, comprising contacting a representative cell with said adenoviral replicon and observing any detrimental effect. Once said replicon has been identified, it can be used to specifically eliminate certain cells involved in disease, for instance tumor cells. Preferably, said replicon contacts, enters and replicates predominantly in diseased cells, causing a detrimental effect in said cells, while in non-diseased cells no or a tolerable detrimental effect is induced. Preferably, said adenoviral replicon comprises a recombinant adenovirus with a fusion between DNA from Ad5 and subgroup B adenoviral DNA. Methods for producing and purifying a replicon according to the invention is also herewith provided.

### FIELD OF THE INVENTION

The invention relates to the field of molecular genetics and medicine. In particular, the present invention relates to the field of gene therapy, more in particular to gene therapy using viral vectors, especially adenoviruses to treat cancer.

### BACKGROUND OF THE INVENTION

On a yearly basis millions of people world-wide suffer from cancer. Although the incidence of a particular form of cancer can vary regionally, breast-, lung-, colon-, prostate-, pancreas-, and bladder cancer are among the cancer types having the highest incidence. With an overall average survival chance of 50%, cancer claims the second highest mortality rate in the world. As the incidence increases with age, its incidence will further increase as the result of an aging population. Treatment for cancer is generally conventional i.e. surgery, radiotherapy, chemotherapy or combinations hereof.

The formation of a tumor is a highly complex process that presumably is initiated through a number of mutations in the DNA of a single cell. The transformation of this single cell is accompanied by the fact that the cell also becomes immortalized resulting in that the cell no longer responds to signals from outside or from neighboring cells to stop proliferating. Moreover, the cell has lost the ability that is normally present in non-diseased cells to go into programmed cell death or apoptosis when mutations in the DNA occur that can no longer be handled by DNA repair mechanisms.

During growth of the tumor it becomes vascularized by formation of blood vessels that are sprouting in the tumor, mainly driven by the cytokine production of the tumor cells. The blood supply provides the tumor with nutrients and oxygen and also allows invasion of tumor cells to other tissues (metastasis).

A relatively novel way to treat cancer is by means of gene therapy. Many gene therapy based strategies to specifically target tumor cells in humans involve the use of DNA delivery vehicles like viruses. One of the major focuses in this field of anti-cancer gene therapies has been on recombinant adenoviruses, since these viruses are well known for their ability to transfer DNA into a cell in a very efficient manner. Recombinant adenoviruses can be produced in large quantities and to very high titers in so-called packaging cells. Moreover, the genome of the, adenovirus leaves enough room to introduce therapeutic genes. Anti-cancer therapies that have been developed to date that involve recombinant adenoviruses are mainly based on adenovirus serotype 2 or serotype 5, since their genomes are well characterized and their life cycle and cell binding properties are well understood.

To date six different subgroups of human adenoviruses have been proposed which in total encompasses 51 different serotypes (De Jong et al. 1999). A serotype is defined on the basis of its immunological distinctiveness as determined by quantitative neutralization with animal derived antisera (horse, rabbit etc). If neutralization shows a certain degree of cross-reaction between two viruses, distinctiveness of serotype is assumed if the hemagglutinins are unrelated as shown by the lack of cross-reaction of hemagglutination inhibition or when substantial biophysical/biochemistry differences in DNA exists (Francki et al. 1991). The nine serotypes identified last (serotypes 42 to 51) were isolated from HIV infected patients (Hierholzer et al. 1988; Schnurr et al. 1993; De Jong et al. 1999). For reasons not well understood, most of such immune-compromised patients shed adenoviruses that were rarely or never isolated for immune-competent individuals (Hierholzer et al. 1988 and 1992; Khoo et al. 1995; De Jong et al., 1998). It should be noted that the Ad51 that is described here is Ad50 in the publication of De Jong et al. (1999). Likewise, Ad50 that is described here is Ad51 in the publication of De Jong et al. (1999).

The recombinant adenoviruses that are used for gene therapy purposes generally do not replicate in human cells, since these adenoviruses lack genes such as the genes in the E1 region that are required for replication. The replication supporting genes are provided only by the packaging cells. These non-replicating recombinant adenoviruses usually contain therapeutic genes in and instead of the E1 region. Due to this, the virus stock is produced in the packaging cell line and subsequently used to infect cells that do not support replication and packaging but instead express the therapeutic genes encoded by the heterologous DNA that is been introduced in the recombinant adenovirus.

Although the adenoviruses seem to be promising tools in gene therapy for the treatment of tumors, an increasing number of disadvantages have become apparent over the last few years. Most adenoviruses that are injected directly into the bloodstream end up in liver cells instead of the tumor cells where their effects are needed. Apparently, the different adenovirus serotypes can differ significantly in their cell type specificity. Furthermore, therapies in which it is necessary to repeat the treatment with a number of doses of the same adenovirus render the immune system of the treated individual to very efficiently neutralize adenoviruses that are injected in following injections.

Another drawback of the use of adenoviruses is that the recombinant adenoviruses used to date only target the outside of the tumor. The cells on the inside of the tumor remain non-infected due to penetration limitations of the recombinant adenovirus. Although with these strategies tumor regression has been observed in many animal models it has not been demonstrated that complete tumor eradication could be obtained in in vivo animal models with a significant tumor mass. The cells on the inside will grow out eventually unless treated differently. These features result in very low efficiencies of this kind of gene therapy strategies and result furthermore in the necessity of very high doses of viruses that are applied to finally obtain some sort of significant therapeutic effect (D'Ambrosio et al. 1982).

### BRIEF DESCRIPTION OF TABLES AND FIGURES

Table A. Adenovirus chimaeric vectors used on different cell lines. Ad5 is the backbone vector, while the Fib vectors are the chimaeric vectors carrying a fiber from the indicated serotype on the Ad5 backbone.

Table B. Comparison between chimaeric vectors carrying the fiber protein from different serotypes and the original serotypes the fiber was derived from. The multiplicity of infection that was used was 10 virus particles per cell.

Table C. Different situations used in example 2.

Table D. Subgroup B adenoviruses used on different cells. Multiplicity of infection: 10 virus particles per cell.

Table E. Subgroup D adenoviruses used on different cells. Multiplicity of infection: 10 virus particles per cell.

Table F. Generation of progeny virus.

FIG. 1. Infection ability of fiber-chimaeric vectors to infect human primary pancreatic carcinoma tumor cells (A) Donor 1, (B) Donor 2, (C) Donor 3, (D) Donor 4.

FIG. 2. Replication ability of adenovirus serotypes as determined by the amount of viable cells present 6 days after the addition of 150 virus particles of replication competent adenoviral serotypes (A) Donor 1, (B) Donor 2, (C) Donor 3, (D) Donor 4.

### SUMMARY OF THE INVENTION

The present invention provides methods for identifying an adenoviral replicon, such as adenovirus capable of efficiently eliminating a target cell, wherein said adenoviral replicon harbors all features essential for said adenoviral replicon to replicate in said target cell, said method comprising the steps of: contacting said target cell or a cell which is representative for said target cell, with an adenoviral replicon; determining whether said adenoviral replicon is capable of infecting said target cell more efficiently than adenovirus serotype 5; and determining whether said target-cell is efficiently eliminated by said adenoviral replicon.

The present invention further provides a method for producing such adenoviral replicons, said method comprising the steps of: providing said identified replicon; culturing a producer cell in a culture medium; contacting said producer cell with said adenoviral replicon; allowing replication of said adenoviral replicon in said producer cell; and obtaining replicated adenoviral replicons from said producer cell and/or said culture medium.

The present invention is especially useful for identifying and producing adenoviral replicons that can be applied for the treatment of human and animal subjects suffering from neoplastic disease such as tumors.

### DETAILED DESCRIPTION

The present invention provides a method for identifying an adenoviral replicon capable of efficiently eliminating a target cell, wherein said adenoviral replicon harbors all features essential for said adenoviral replicon to replicate in said target cell, said method comprising the steps of: contacting said target cell with an adenoviral replicon; determining whether said adenoviral replicon is capable of infecting said target cell more efficiently than adenovirus serotype 5; and determining whether said target cell is efficiently eliminated by said adenoviral replicon. In another embodiment the invention provides a method for identifying an adenoviral replicon capable of efficiently eliminating a target cell, wherein said adenoviral replicon harbors all features essential for said adenoviral replicon to replicate in said target cell, said method comprising the steps of: contacting a cell which is representative for said target cell, with an adenoviral replicon; determining whether said adenoviral replicon is capable of infecting said representative cell more efficiently than adenovirus serotype 5; and determining whether said representative cell is efficiently eliminated by said adenoviral replicon. Said adenoviral replicon is identified if it is capable of infecting said target cell, or said representative cell, more efficiently than adenovirus serotype 5, and if said target cell or said representative cell is efficiently eliminated by said adenoviral replicon as well.

Said target cell may also be used as a representative cell when said cell is obtained from tissue, such as tumor tissue, and used in settings to have the adenoviral replicon replicate and eliminate said target cell. Said target cell is then representative for all cells present in the (tumor) tissue from which the target cell was derived. In one embodiment said representative cell is a transformed brain cell, gastro-intestinal cell, pancreatic cell, liver cell, breast tumor cell, cervix cell, lung cell or skin cell. In another aspect a representative cell is not derived directly from (tumor) tissue but is a cell of an indefinite growing cell line, a transformed cell, or a cell that has characteristics that are similar and/or representative for said target cell. A representative cell is not necessarily from the same species as the target cell. It is however preferred that said target cell and said representative cell are human.

In a preferred aspect of the invention said adenoviral replicon comprises an adenovirus and it is even more preferred that said adenovirus comprises a recombinant adenovirus and/or a chimaeric adenovirus. It is preferred that said adenovirus comprises an alteration in its E1 region, such as in E1B and/or in its. E3 region. Alterations as used herein means deletion of nucleic acid, swapping of parts of the nucleic acid, point mutations, insertions, and/or chemically induced mutations of the nucleic acid. These alterations may result in (total or partly) functional loss of the encoded protein(s), and/or may result in frame-shifts that might result in the termination of the encoded protein(s). In one preferred embodiment said alteration in said adenovirus results in loss of at least part of E1B-55K function. This is particularly useful since adenoviruses that-have a loss of E1B-55K function can only replicate in p53-minus cells, which mean s that normal cells that express a normal (non-tumor inducing p53 protein) cannot support the adenovirus that harbours this E1B-55K alteration. In this sense, the adenoviral replicon is only capable of replicating in certain kinds of cells (e.g, tumor cells expressing a mutant p53 or no p53) whereas in other kinds of cells (expressing wild type p53) the adenoviral replicon cannot replicate. It is therefore preferred that said target cell does not efficiently express a functional p53 protein. It is also preferred that said target cell is a neoplastic cell such as a tumor cell.

The invention also provides a method, wherein said adenoviral replicon is derived from subgroup B and/or C adenovirus, and wherein said adenoviral replicon is not wild type adenovirus serotype 5. Preferably, said adenoviral replicon is derived from adenovirus serotype 7, 11, 14, 21 or 51. Adenovirus serotype 5 is one of the best-studied adenoviruses in the art. However, this adenovirus tends to home to the liver instead of the specific target cells, when the wild type virus or the recombinant virus that is-derived or based on adenovirus serotype 5 is injected intraveneously. There is a well-recognized need in the art for specific targeted adenoviruses.

The present invention further provides a method, wherein said adenoviral replicon comprises nucleic acid comprising a fusion between nucleic acid from adenovirus serotype 5 and nucleic acid from an adenovirus serotype from subgroup B. In another embodiment the invention provides a method, wherein said adenoviral replicon comprises nucleic acid comprising a fusion between at least a part of a structural protein encoding nucleic acid from a first adenovirus serotype and at least a part of a structural protein encoding nucleic acid from a second adenovirus serotype. It is preferred that said first adenovirus serotype is adenovirus serotype 5 and said second adenovirus serotype is an adenovirus serotype from subgroup B. More preferably, said second adenovirus serotype is adenovirus serotype 13, 16, 35 or 51. In a preferred embodiment of the invention said structural protein encoding nucleic acid encodes a fiber protein. The fiber protein is known to be involved in receptor recognition and/or cell entry. It is therefore very useful to modify the fiber protein for specific targeting.

In another aspect, a method of the invention is provided wherein said adenoviral replicon comprises an alteration in a gene encoding a protein involved in replication. Such alterations may render the adenoviral replicon to replicate in a cell-specific manner or to enhance replication in itself.

It is further preferred that the methods of the invention are carried out in a high-throughput format. Persons skilled in the art will appreciate that the present invention is useful in settings wherein sets of adenoviral replicons, being wild type, recombinant and/or chimaeric are tested in large settings wherein different kinds of representative or target cells are tested to identify the most suitable replicating adenoviral replicon that eliminates the target cell and/or the representative cell in the most efficient manner, such as high-throughput settings. Efficient elimination as used herein means that the adenoviral replicon is capable of killing the infected cell within a reasonable amount of time. This elimination may be through direct lysis of the cell or through the induction of detrimental effects such as apoptosis. Since apoptosis may be a relatively long process it is preferred that said elimination comprises lysis.

In another embodiment the invention provides a method for producing an adenoviral replicon identified by a method of the invention, said method for producing an adenoviral replicon comprising the steps of: providing an adenoviral replicon identified by a method of the invention; culturing a producer cell in a culture medium; contacting said producer cell with said adenoviral replicon; allowing replication of said adenoviral replicon in said producer cell; and obtaining replicated adenoviral replicon from said producer cell and/or said culture medium. In one aspect of the invention said producer cell comprises a functional E1 region obtainable from adenovirus. Preferably said producer cell is a PER.C6, A549 or U87 cell or a,derivative thereof.

The invention further provides a pharmaceutical composition comprising an adenoviral replicon, identified and/or obtainable by a method of identifying and/or producing an adenoviral replicon of the invention. Said adenoviral replicon can be applied for use in treating a human or animal body by surgery, therapy or diagnostics. A pharmaceutical composition of the present invention is particularly useful for the treatment of neoplastic disease. Through the identification of said adenoviral replicon, the invention also provides a method for treating a neoplastic condition, comprising administering a composition comprising an adenoviral replicon capable of eliminating a neoplastic cell to an individual having a neoplasm. It is preferred that said neoplasm comprises a tumor.

The present invention provides means and methods for the identification, the production, the purification and the use of an adenoviral replicon capable of eliminating a target cell. In one aspect the invention provides a method for identifying an adenoviral replicon capable of eliminating a target cell, comprising contacting a representative cell with said adenovirus replicon and observing any detrimental effect. An adenoviral replicon comprises an adenoviral nucleic acid having all necessary elements for replication of said nucleic acid in a cell. Elements for replication of said nucleic acid may partly be present in trans. In one embodiment said adenoviral replicon comprises an element allowing packaging of said adenoviral replicon into a virus-like particle. Non-limiting examples of an adenoviral replicon are wild type adenoviruses. In another embodiment said adenoviral replicon comprises an element allowing said adenoviral replicon and/or said virus-like particle to contact and to enter a cell. In another embodiment said adenoviral replicon and/or said virus-like particle is capable of contacting and entering a tumor cell. In another embodiment said adenoviral replicon and/or said virus-like particle predominantly replicates in tumor cells. A representative cell is defined as a cell that indicates which adenoviral replicon has, or has a more pronounced, detrimental effect on a certain kind of aberrant cell. Preferably said representative cell comprises a human cell. Human cells are better predictors for human disease than non-human cells. Preferably, said representative cell comprises a tumor cell. Adenoviral replicons capable of conferring detrimental effects on said representative cell can be used to treat tumor types. Said tumor types comprise at least those tumor types that said representative cell is a derivative or analogue of. Non-limiting examples of representative cells are A549 (Human lung epithelia, ATCC CCL-185), Capan-1 (Human pancreas, ATCC HTB-79), MCF-7 (Human breast, ATCC, HTB-22), BxPC3 (Human pancreas, ATCC, CRL-1687), U87 (Human astroglioma ECACC no.89081402), SK-N-MC (Human neuroblastoma, ATCC HTB-110), MIA-PaCa-2 (Human pancreas, ATCC CRL-1420), Hs766T (Human pancreas, ATCC HTB-134), HeLa (Human Cervix, ATCC, CCL-2), HepG2 (Human liver ECACC no.85011430), SK-Cha-1 and Mz-Cha-1 (Both human cholangio carcinoma), OE19 and OE33 (Both human oesophagus ECACC no.96071721 and 96070808 resp.) and TE-1, TE-2 and T-tn (All three human squamous cell carcinoma).

In one aspect the present invention provides a wild type or recombinant adenoviral replicon such as an adenovirus capable of specifically causing a detrimental effect in a diseased cell such as a tumor cell, while in a non-diseased cell no or a tolerable detrimental effect is induced. In one embodiment said specificity is provided by a specific recognition of target cells, a specific replication in target cells and/or a specific infection of said target cell. The invention provides the use of said adenoviral replicon in the production of a pharmaceutical composition and the use of said pharmaceutical composition for gene therapy purposes. According to the invention said adenoviral replicons can be of wild type origin or are made recombinant and/or chimaeric by modifications in the nucleic acid. A chimaeric adenovirus as used herein-means that the adenovirus comprises modifications in the nucleic acid such that nucleid acid is derived from at least two different serotypes. Derived as used herein for nucleic acid means for example direct cloning from a wild-type adenovirus serotype or through nucleic acid multiplication via for instance the polymerase chain reaction used commonly by persons skilled in the art of molecular biology. Examples of modifications in the DNA of said adenoviral replicons or said adenoviruses are modifications in the genes encoding capsid proteins or in genes encoding proteins involved in replication. Preferably said capsid protein is a fiber protein of adenovirus. The fiber protein is involved in receptor recognition and specificity for cellular tropism. It is well recognized in the art that several adenoviruses, like for instance adenovirus serotype 5, cannot be used to target specific (subsets) of cells since they home directly to the liver. It is one aspect of the invention to use the fiber of a first adenovirus and make a chimaeric adenovirus with a second adenovirus to change the tropism of the second serotype and infect the cells that need to be targeted and that would otherwise not be targeted efficiently by said second adenovirus. As used herein, by a replicon capable of infecting a target cell more efficiently than adenovirus subtype 5 is meant that the infection characteristics of said replicon towards said target cell are more favorable than the infection characteristics of adenovirus subtype 5. This can for instance be due to a higher affinity of said replicon for said target cell as compared to adenovirus serotype 5. Said replicon can also have less tendency to home directly to the liver. In that case, said replicon is better available for other cells. Said less tendency to home directly to the liver can render the infection characteristics of said replicon towards said target cell more favorable, even though the affinity of said replicon for said target cell may be lower than the affinity of adenovirus serotype 5 for said target cell. A recombinant adenovirus as used herein does not need to be a chimaeric adenovirus, although a chimaeric adenovirus is by definition a recombinant adenovirus and not a wild type adenovirus. The recombinant adenovirus may also be derived from one or more adenovirus serotypes but may also harbor different kinds of alterations in its nucleic acid. In a preferred embodiment the invention provides methods according to the invention wherein said adenoviral replicon comprises heterologous nucleic acid, such as a nucleic acid encoding a tumor-specific antigen or a functional equivalent thereof. Heterologous nucleic acid as used herein means any nucleic acid that is not present in the wild type adenoviral replicon in which the heterologous nucleic acid is introduced. Therefore, the heterologous nucleic acid as used herein may be from a non-adenoviral replicon source, but may also comprise a. nucleic acid that is swapped between different adenoviral replicons. Non-limiting examples of heterologous nucleic acid that can be introduced in the adenoviral replicons are nucleic acids encoding tumor antigens, apoptosis inducing proteins, anti-angionic proteins, lysis-inducing proteins and other proteins that are toxic/detrimental for the target cell and antisense nucleic acids. Non-limiting examples of tumor antigens that can be inserted are gp100, CAMEL, PRAME, Mart-1, Mage-1, Melan-A and tyrosinase. In another preferred embodiment, said heterologous nucleic acid comprises a nucleic acid encoding a detrimental effect inducing protein or a functional equivalent thereof, such as VP3 from chicken anemia virus, cytosine deaminase, nitroreductase, thymidine kinase and linamarase. In yet another preferred embodiment said heterologous DNA comprises a nucleic acid encoding a protein capable of inducing anti-angiogenic effects, or a functional equivalent thereof, such as a VEGF antagonist or ATF-BPTI. To inhibit any effect within a diseased cell such as a tumor cell-said heterologous nucleic acid can also be present in an antisense form. Antisense as used herein means that the transcription product of said heterologous nucleic acid may inhibit their counterparts (the endogenously present cellular product, present in the cell that was infected by the adenoviral replicon) for instance through hybridization.

In another aspect the invention provides a method for producing an adenoviral replicon of the invention comprising contacting a producer cell with said adenoviral replicon and obtaining produced adenoviral replicon from said producer cell and/or culture medium. Said producer cell enables the generation of very high titers of said adenoviral replicon in said producer cell. Upon lysis of said producer cell produced adenoviral replicons become present in said culture medium, from which said produced adenoviral replicons are purified. In another aspect the invention provides a method for producing an adenoviral replicon of the invention comprising contacting a producer cell with said adenoviral replicon, wherein said producer cell comprises a representative cell or derivative thereof. A representative cell that generates significant high titers of said adenoviral replicon for the generation of pharmaceutical compositions for the use in the treatment of aberrant cells is preferably used as a producer cell for said adenoviral replicon. In another aspect the invention provides a method for producing an adenoviral replicon of the invention comprising contacting a producer cell, wherein said producer cell comprises a functional E1 region derived from adenovirus. Preferably said producer cell comprises a PER.C6 cell or a derivative thereof. In another aspect the invention provides a method for producing an adenoviral replicon of the invention comprising contacting a producer cell, wherein said producer cell comprises a A549 or U87 cell or a derivative thereof.

The invention provides the use of a producer cell such as a PER.C6, A549 and U87 cell or another representative cell that supports the replication and packaging of said wild type, recombinant and/or chimaeric adenoviral replicons and adenoviruses. The generation of said adenoviral replicons in said producing cells results in significant titers of adenoviral replicons that can be used in pharmaceutical compositions that can be used for the treatment of aberrant or neoplastic cells such as tumor cells.

Adenoviral replicons provided by the invention can replicate in tumor cells and in representative cells resulting in a detrimental effect such as apoptosis and/or lysis of said tumor cells. Importantly, a detrimental effect can also be the induction of necrosis, cell cycle arrest, DNA fragmentation, apoptosis or lysis in tumor cells present in said solid tumors that were not infected during the first administration. This results in eradication and removal of a larger tumor mass than could be obtained by using non-replicating recombinant adenoviruses that only target cells on the outside of the tumor. Lysis of cells that were not transduced during the first administration is due to the infection, replication and packaging of the viruses that were produced in the cells that were infected at the first administration. This results in a deeper penetration and a therapeutic effect that is deeper into the tumor than was obtained thus far with recombinant non-replicating adenoviruses. The present invention further provides diminished replication ability in non-tumor cells of the wild type or recombinant adenoviruses provided by the invention.

The recombinant adenoviruses provided may carry mutations in the E1 and/or E2 and/or E3 regions of the adenoviral genome. The mutations in the E1 region preferably are in the E1A and/or E1B gene. In one embodiment the invention provides the use of an adenoviral replicon preferably a recombinant adenovirus in which the E1 region of the adenovirus is modified in such a way that said adenoviral replicon or adenovirus replicates more efficiently in tumor cells than in non-tumor cells. The modifications provided by the present invention involve alterations like deletions, point mutations and/or additions. In another embodiment the present invention provides the use of a mutation in the E1B 55K encoding gene that enables the specific replication in tumor cells that lack functional tumor suppressor proteins like p53.

The invention also provides the use of recombinant chimaeric adenoviral replicons or adenoviruses of the invention in which the capsid derived from adenovirus has been modified to specifically target a diseased cell such as a tumor cell. The modification of the capsid may involve the exchange of the genes encoding fiber and/or hexon and/or penton. In one embodiment of the present invention a recombinant adenoviral replicon or adenovirus is built up from parts from different serotype subgroups. In another embodiment of the invention a gene encoding the fiber protein of adenovirus serotype 5 (subgroup C) is (partly) replaced by (part of) a gene encoding the fiber protein from another serotype. In an even more preferred embodiment (part of) a gene encoding the fiber protein of adenovirus serotype 5 is replaced by (part of) a gene encoding the fiber protein from an adenoviral serotype from subgroup B such as Ad11, Ad 13, Ad16, Ad35 and Ad51.

The present invention further provides adenoviral replicons or adenoviruses harboring mutations in regions that are involved in replication such as the Inverted Terminal Repeats (ITR's) that are present on both ends of the viral genome and the genes encoding E2 proteins, the precursor terminal protein pTP and the DNA polymerase pol protein. These mutations result in a more specific replication in aberrant cells such as tumor cells as compared to non-aberrant cells.

In yet another embodiment of the present invention the adenoviral genome is modified by mutations in regions harboring genes that are necessary for the adenovirus to replicate and escape a defense mechanism of the transduced cell and to escape at least part of the immune system of the treated individual. In a more preferred embodiment the invention provides an adenovirus with a mutation in the E1 region and/or the E2 and/or the E3 region besides point mutations and/or deletions in other regions of the adenoviral genome to enhance replication specificity in tumor cells.

The invention further provides the selection and use of recombinant adenoviral replicons or adenoviruses that have incorporated heterologous DNA in regions in the genome, wherein said regions are not involved in replication processes. In one embodiment of the present invention the heterologous DNA is incorporated in the E3 region. Because E3 is involved in immune suppression, the replicating virus expresses proteins with by stander effects and adds to the enhancement of immune responses from the treated individual towards the infected tumor cell. In another embodiment heterologous DNA that is incorporated in the adenoviral genome such as the E3 region encodes for antigens such as gp100, CAMEL, PRAME, Mart-1, Mage-1, Melan-A, tyrosinase (Kawakami 2000, Tsukamoto et al. 2000, Jager et al. 2000, Mackensen et al. 2000, Yang et al. 2000, Tuting et al. 1999, Aarnoudse et al. 1999) or parts thereof. In another preferred embodiment heterologous DNA encodes an antigen that will stimulate the immune system of the treated individual to target the infected tumor cells but also non-infected tumor cells. Heterologous DNA that is incorporated according to this specific aspect of the invention can encode proteins such as interleukins, preferably IL-2, IL-3 and/or IL-6. Other immune system stimulatory genes such as GM-CSF are incorporated in the adenoviral genome. Typically a cell that is transduced starts producing the encoded proteins that subsequently stimulate cells from the immune system such as natural killer cells (NK), T-cells, and macrophages to remove transduced and non-transduced tumor cells.

In another embodiment of the present invention an adenoviral replicon of the invention comprises heterologous DNA involved in induction of apoptosis to not only lyse the cells by the fact that the adenoviral replicon or adenovirus is replicating, packaged and released, but also to stimulate surrounding cells to exhibit detrimental effects such as apoptosis. In this specific part of the invention adenoviral replicon or adenovirus renders two separate killing actions: One being the lysis of the infected cells due to the replication, production and packaging of the virus particles; another being through the by-stander effect of the death inducing proteins encoded by the heterologous DNA such as the VP3 protein from chicken anemia virus, the cytosine deaminase protein, the nitroreductase protein, the thymidine kinase protein from herpes simplex virus, the linamarase protein (Pietersen et al. 2000; Stackhouse et al. 2000; Koyama et al. 2000; Weedon et al. 2000; Cortes et al. 1998) or a functional equivalent thereof on surrounding cells.

In another embodiment of the invention replicating adenovirus comprises heterologous DNA encoding one or more proteins or functional equivalents thereof that have anti-angiogenic effects such as ATF-BPTI and some VEGF antagonists (Inoue et al. 2000; Gagnon et al. 2000). As a result of this, expressed protein(s) will inhibit further growth of blood vessels into tumor and thereby prevent further outgrowth of tumor cells and lower the possibility of a tumor to metastasize. Heterologous DNA can be incorporated in regions that are not involved in the replication machinery of the virus. Heterologous DNA can also be present in combinations with alterations in the viral genome that enhance the replication capability of said virus in tumor cells.

### EXAMPLES

### Example 1

Adenovirus Entry and Replication on Human Tumor Cell Lines.

To compare the replication efficiency of different adenovirus serotypes it was investigated whether tumor cells support the entry of a particular adenovirus. Hereto, a small panel of human cancer cell lines was transduced with Ad5-based vectors carrying the fiber molecule derived from alternative serotypes. Human cancer cell lines tested for the susceptibility of different fiber chimaeric vectors were: A549 (Human lung epithelia, ATCC, CCL-185), Capan-1 (Human pancreas, ATCC, HTB-79), MCF-7 (Human breast, ATCC, HTB-22), BxPC3 (Human pancreas, ATCC, CRL-1687), U87 (Human astroglioma ECACC ref no. 89081402), SK-N-MC (Human neuroblastoma, ATCC, HTB-110), MIA-PaCa-2(Human pancreas, ATCC, CRL-1420), Hs766T (Human pancreas, ATCC, HTB-134), HeLa (Human Cervix, ATCC, CCL-2), HepG2 (Human liver ECACC ref no. 85011430), SK-Cha-1 and Mz-Cha-1 (Both human cholangio carcinoma, gift from Dr. Knuth, Frankfurt, Germany), OE19 and OE33 (Both human oesophagus-ECACC ref no. 96071721 and 96070808 respectively), and TE-1, TE-2 and T-tn (All three human squamous cell carcinoma, gift from Dr. Heideman, Amsterdam, The Netherlands). Of each cell line, 1 cells were seeded in wells of 24-well plates in Dulbecco's modified Eagles medium (DMEM) supplemented with 10% fetal calf serum (FCS). 24 h later, cells were exposed to a virus dose of 1000 virus particles per cell of each of the chimaeric vectors indicated in Table A. 48 h after virus addition cells were washed twice with 1 ml PBS after which cells were lysed by adding 100 μl of lysis buffer. Luciferase transgene activity was determined using a bioluminescence machine and the luciferase assay kit from Promega (catalog nr. E-1501) following the instructions provided by the manufacturers. The results shown in Table A reflect the ability of the different adenoviral vectors to enter the tumor cell. Also shown in Table A are the results obtained after screening each cell type for the presence of the Coxsackie adenovirus receptor (CAR) which is the primary receptor for adenovirus serotype 5. The results of these experiments thus show that Ad5 and the chimaeric fiber viruses selected are able to enter all tumor cells tested albeit with different efficiencies.

### Example 2

Influence of Virus Entry.

In a next experiment the influence of virus entry on the ability of wild type adenoviruses to replicate in a panel of human cell lines was tested. As a positive control for virus viability, PER.C6 cells were taken since all viruses could be generated on this human cell line. Ad5 vectors carrying the fiber molecule of several other adenovirus serotypes, i.e., Ad13, Ad16, Ad35, and Ad51 were generated. For details regarding the cloning strategies and the production of the recombinant viral vectors see WO 99/55132, WO 00/03029, WO 00/52186 and WO 00/70071. Virus was added at a dose of 10 virus particles/cell on different cell lines and the presence and robustness of CPE was scored 6 and 10 days after virus administration. The results of these experiments are shown in Table B. To interpret these results and discriminate between virus entry and replication the scheme as depicted in Table C was developed.

The situations numbered 2, 3, 7, and possibly 5, 6, and 8 in Table C (depending on the onset and robustness of Cyto Pathological Effect (CPE)) could result in vectors that are better suited to use as recombinant vectors for cancer cell line transduction and replication. Situation (2) on a certain cell line could mean that another fiber on Ad5 is an improvement over Ad5. Situation (3) on a certain cell type could mean that another serotype must be used instead of Ad5. Situation (5) on a certain cell type could mean that another serotype might be better compared to Ad5, depending on the time of onset and robustness of CPE. Situation (6) on a certain cell type could mean that another fiber on Ad5 might be better compared to Ad5, depending on the time of onset and robustness of CPE. Situation (7 and 8) on a certain cell type could mean that either another serotype or another fiber might be better compared to Ad5.

Situation (1): non-conclusive since the lack of CPE observed with both adenoviruses can be due to either entry or the ability to replicate on such a cell line. Examples of-this situation are U87, MiaPaca-2, and SK-N-MC.

Situation (2+6): either Ad5 or the fiber-modified Ad5 vector gives CPE. In situation (2) Ad5 can probably replicate but not enter whereas AdX can enter but not replicate. In situation (6) a fiber-modified vector is better as compared to Ad5 if a higher CPE score is found. Examples of these situations are found with all, fiber-modified vectors on Hs776T, and for Ad5/Ad35/Ad5Fib35 on MCF-7.

Situation (3+7): either AdX or Ad5FibX gives CPE. In situation (3) another serotype is required to obtain replication in a certain cancer cell line. This situation was not observed using this relatively small panel of human cell lines. In situation (7) Ad5 and AdX can both replicate, but entry of Ad5 is impaired. This situation was not observed using this relatively small panel of human cell lines.

Situation (4): only Ad5 gives CPE, thus states that AdX can at least not enter the cell since Ad5FibX cannot replicate. An example of this situation is found for Ad5/Ad13/Ad5Fib13 on U251. Here, Ad5 is superior compared to the other vectors and fiber-modified viruses tested.

Situation (5): both Ad5 and AdX give CPE but not Ad5FibX is unlikely to exist and is also not observed.

Situation (8): similar to situation 7 however Ad5 itself is able to replicate. A better vector compared to Ad5 depends on the time of onset and robustness of CPE. Examples of this situation are found for Ad5/Ad35/Ad5Fib35 on Capan-1 and BxPC3, or for Ad5/Ad16/Ad5Fib16 on Capan-1.

The results using this panel of human cancer cell lines show that either another adenovirus serotype or exchange of the fiber molecule of Ad5 for a fiber molecule from another serotype can result in a vector which has a better entry and/or replication advantage. Interestingly, on several human cancer cell lines it was observed that all B-group derived-vectors tested (16, 35, 51) did as good as or better than Ad5. This experiment was repeated, thereby taking all subgroup B adenovirus wild types (except 51). In this experiment CPE was determined at 3 days and 6 days after virus exposure (10 virus particles per cell). The results of this experiment are shown in Table D. These results show that except for U251, B-group serotypes replicate better compared to Ad5 on all human cancer cell lines tested which represent human pancreas, breast, and lung tumor cell lines.

### Example 3

Replication of Subgroup D Viruses.

Subgroup D adenoviruses were compared with Ad5 for their ability to replicate on human tumor cells. The experiment was performed as described above (10 virus particles of each virus per cell) and CPE was scored on day 3 and day 6. The results of these experiments are, shown in Table E. In contrast to the positive control PER.C6 and as compared to A549, many of the p-group adenoviruses (21 out of 33 tested=67%) replicate very poorly on the selection of human tumor cells. It is important to note that all cell lines were infected simultaneously with the same adenoviral batches, indicating that small differences in the quality of virus batches due to different treatments such as purifications or storage conditions cannot account for the observed differences in onset or robustness of CPE.

### Example 4

Generation of Progeny Virus.

For the spread of virus through a tumor mass it is important that progeny virus is formed. When scoring CPE one scores for the toxicity of the virus infection and not for progeny formation although clear CPE usually indicates a lytic virus infection accompanied with release of progeny. To investigate whether virus is indeed generated with the formation of CPE the cells were harvested when CPE had occurred of some cell lines and of some viruses. Cells were frozen and centrifuged for 5 min at 1750 rpm. Of the supernatant 250 μl was added to 2.75 ml DMEM/10% FCS. This 3 ml was used to infect a new layer of tumor cells and the formation of CPE scored. The results of this experiment are shown in Table F. These results show that life virus progeny is generated.

### Example 5

Infection Ability of Fiber-Chimeric Vectors for Human Primary Pancreatic Tumor Cells.

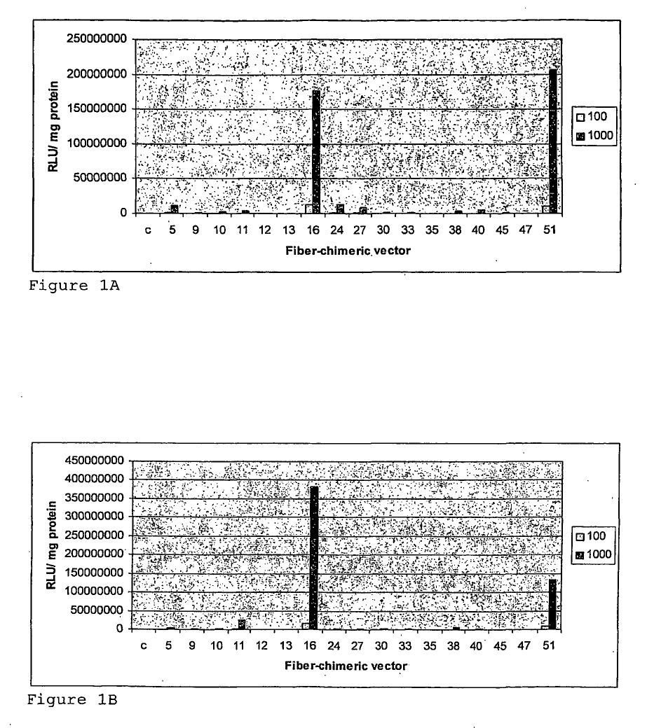

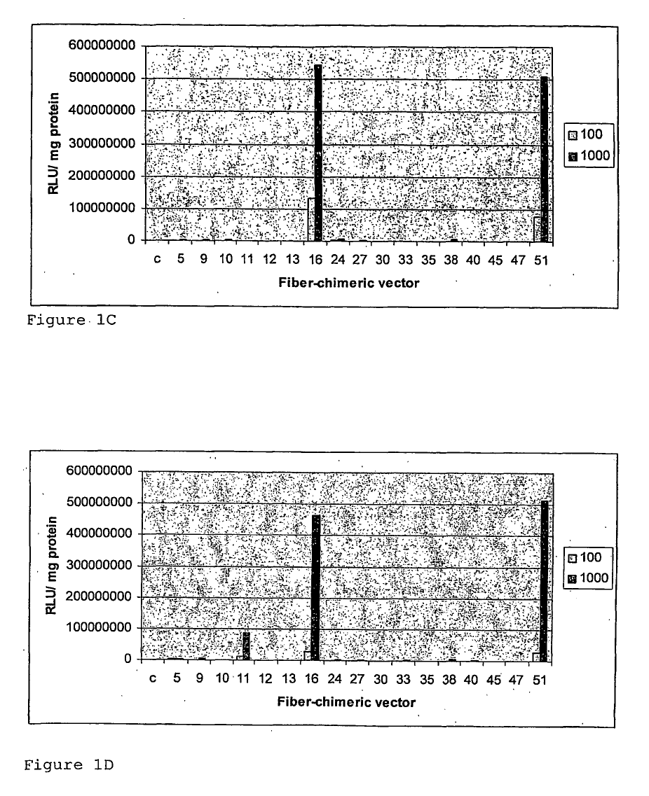

To determine whether some fiber-chimaeric vectors present in the library are suited to infect primary human tumor cells, single cell suspensions were generated from 4 different pancreas carcinoma tumors. To obtain a single cell suspension from a tumor mass, first the tumor mass was cut into small fragments using sterile scissors, spatula, and tweezers. Next, the total-weight of the tumor mass was determined. For each gram of tumor mass 1 ml of a 0.1% collagenase solution was added to obtain single cells. Tubes containing these tumor mass/collagenase mixtures were subsequently placed (with screw cap on) in a 37° C. water bath for 90-120 min. After this, small lumps of tumor mass were separated from the single cell suspension by sieving the complete supernatant through sterile cloth. The sieve was washed twice with 5 ml of sterile phosphate buffered saline (PBS). The total volume of single cell suspension was centrifuged for 5 min at 1500 rpm at room temperature. Supernatant was removed and the cell pellet was dissolved in 5 ml PBS. 20 ml pre-chilled erythrocyte lysis buffer was added to this cell suspension (lysis buffer=1.55 mM NH₄Cl, 11.9 mM NAHCO₃, 0.1 mM EDTA) and this mixture was incubated on ice for 20 min. After this, the suspension was centrifuged for 10 min at 4° C. at 1500 rpm. The cell pellet was resuspended in 5 ml of PBS and again centrifuged for 10 min at 1500 rpm to discard residual lysis buffer. Finally the cell pellet was dissolved in 2.5 ml medium (RPMI+10% FCS). Cell concentration was determined using counting chambers and a microscope. The cell suspension was then diluted to a concentration of 10⁶ cells/ml medium and seeded at concentrations of 105 cells/well in 24-well tissue culture plates. Cells were allowed to recover for 48 h at 37° C. and 5% CO₂. Cells were subsequently exposed to either 100 or 1000 virus particles/cell of each of the fiber-chimaeric vectors carrying the luciferase reporter gene. 48 h after the addition of virus, medium was discarded, cells were washed once with 1 ml of PBS and luciferase reporter activity was determined as described in example 1. The results from 4 different primary pancreatic tumor cell isolations (derived from 4 different donors) are shown in FIG. 1. From these results it can be concluded that two of the fiber-chimaeric vectors are better able to infect primary pancreatic carcinoma cells as compared to Ad5, namely Ad5Fib16 and Ad5Fib51. These chimaeric viruses show increased luciferase activity as compared to the Ad5 vector from which the backbone originated.

### Example 6

Ability of Adenoviral Serotypes to Replicate on Primary Human Tumor Cell.

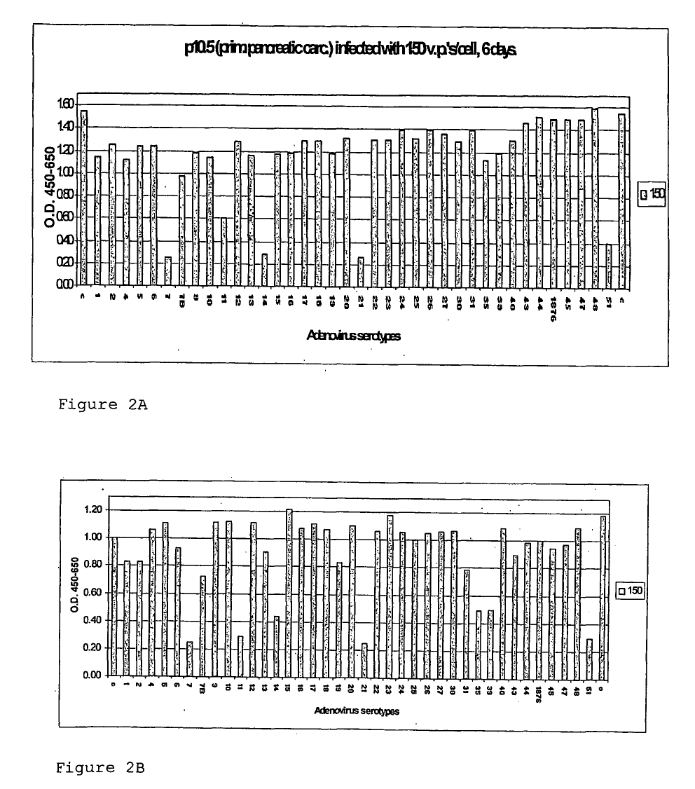

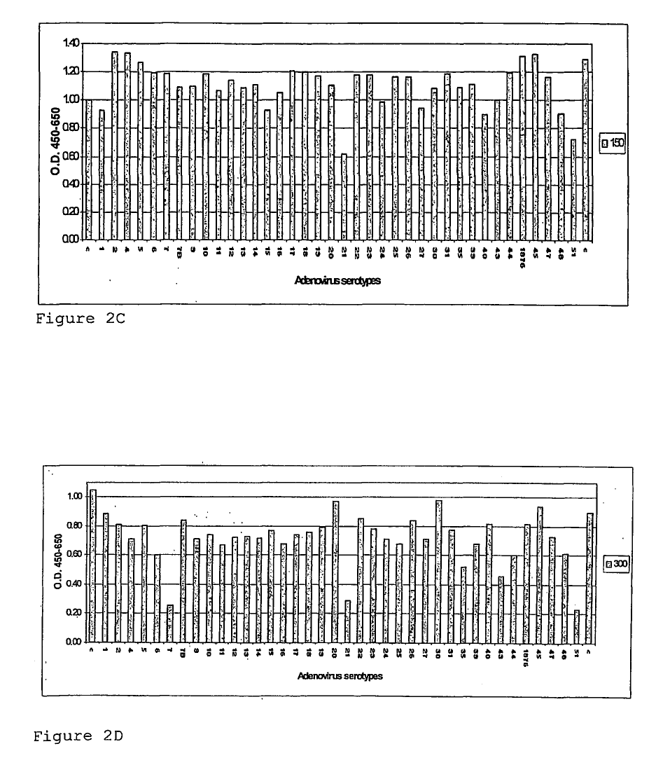

To test whether different adenovirus serotypes differ in their ability to replicate on human primary tumor cells an experiment was set-up using the same cells as described under example 5. However, for these experiments 5×10³ cells were seeded per well in 96-well tissue culture plates and cells were allowed to attach for 24 h at 37° C. and 5% CO₂. Then, 100 μl medium (DMEM+10% FCS) containing 4.5×10⁷ virus particles of different adenovirus serotypes (150 virus particles/cell) were added to each well. At day 6 after virus addition, medium was discarded and cells are incubated for 1 h with 100 μl fresh medium only. After this incubation 10 μl WST-1 solution (Roche) was added to each well and plates were placed at 37° C. and 5% CO₂ for another 30 min. Wells that stained brown to dark-brown contain many viable cells. Wells that stained light to orange-red contain many dead cells (indicative for virus replication). Read-out of the amount of viable cells was performed with an ELISA reader whereby the OD₄₅₀ is corrected for the background by subtracting OD₆₅₀. Results of these experiments are shown in FIG. 2 for 4 different donors respectively. Low OD₄₅₀-OD₆₅₀ values indicate significant cell death and thus significant virus replication. Controls, abbreviated with “c”, were added and indicate the viability of the cell culture at day 0 (first bar) as well as viability of the, cell culture at day 6 (last bar). Based on these results it is concluded that there are differences between donors of primary pancreatic tumor cells, with regard to the ability of adenovirus serotypes. However there is a clear consensus within the performed experiments, that demonstrates that human subgroup B viruses (7, 14, 21, 51) perform significantly better as compared to Ad5 (from subgroup C) on these primary tumor cells. Thus, the ability of adenovirus serotypes to replicate on primary human tumor cells differs and several human adenovirus B-group members have now been identified as more potent as compared to Ad5 to replicate in these cells whereby Ad21 (albeit closely followed by serotypes Ad7 and Ad51), is the most potent serotype.

References

Aarnoudse C A, van der Poul P B, Heemskerk B, Schrier P I (1999). Interleukin-2 induced, melanoma specific T cells recognize CAMEL, an unexpected translation product of LAGE-1. Int J Cancer 82:442-448

Cortes M L, De Felipe P, Martin V, Hughes M A, Izquierdo M (1998). Succesful use of a plant gene in the treatmentt of cancer in vivo. Gene Ther 5:1499-1507

D'Ambrosio E, Del Grosso N, Chicca A, Midulla M (1982). Neutralizing antibodies against 33 human adenoviruses in normal children in Rome. J Hyg Camb 89:155-161

De Jong J C, Wermenbol, A G, Verweij-uijterwaal M W, Slaterus K W, Werthheim-van Dillen P, van Doornum G J J, Khoo S H, Hierholzer J C (1999). Adenoviruses from human immunodeficiency virus-infected individuals, including two strains that represent new candidate serotypes Ad50 and Ad51 of species B1 and D respectively. J Clin Microbiol 37:3940-3945.

Francki R I B, Fauquet C M, Knudson D L, Brown F (1991). Classification and nomenclature of viruses. Fifth report of the international Committee on taxonomy of viruses. Arch Virol Suppl 2:140-144

Gagnon M L, Bielenberg D R, Gechtman Z, Miao H Q, Takashima S, Soker S, Klagsbrun M (2000). Identification of a soluble neurophilin-1 that binds Vascular endothelial growth factor: in vivo expression and antitumor activity. Proc Natl Acad Sci USA 97:2573-2578

Hierholzer J C, Wigand R, Anderson L J, Adrian T, Gold J W M (1988). Adenoviruses from patients with AIDS: a plethora of serotypes and a description of five new serotypes of subgenus D (types 43-47). J Infect Dis 158:804-813

Hierholzer J C (1992). Adenovirus in the immunocompromised host. Clin Microbiol Rev 5:262-274.

Inoue T, Kibata K, Suzuki M, Nakamura S, Motoda R, Orita K (2000). Identification of a vascular endothelial growth (VEGF) antagonist, sFlt-1, fro ma human hemopoietic cell line NALM-16. FEBS 469:14-18

Jager E, Maeurer M, Hohn H, Karbach J, Jager D, Zidianakis Z, Bakhshandeh-Bath A, Orth J, Neukirch C, Necker A, Reichert T E, Knuth A (2000). Clonal expansion iofMelan-A-specific cytotoxic T lympho-cytes in a melanoma patient responding to continued immunization with melanoma-associated peptides. Int J Cancer 86:538-547

Kawakami Y (2000). New cancer therapy by immunomanipulation: development of immunotherapy for human melanoma as a model system. Cornea 19:2-6

Khoo S H, Bailey A S, De Jong J C, Mandal B K (1995). Adenovirus infections in human immunodeficiency virus-positive patients: Clinical features and molecular epidemiology. J Infect Dis 172:629-637

Koyama F, Sawada H, Fujii H, Hirao T, Ueno M, Hamada H, Nakano H (2000). Enzyme/prodrug gene therapy for human colon cancer cells using adenovirus-mediated transfer of the escherichia coli cytosine deaminase gene driven by a CAG promoter associated with 5-fluorocytosine administration. J Exp Clin Cancer Res 19:75-870

Mackenzen A, Herbst B, Chen J L, Kohler G, Noppen C, Herr W, Spagnoli G C, Cerundolo V, Lindemann A (2000). Phase I study in melanoma patients of a vaccine with peptide-pulsed dendritic cells generated in vitro from CD34(+) hemopoietic progenitor cells. Int J Cancer 86:385-392

Pietersen A, Noteborn H M (2000). Apoptin Adv Med Biol 465:153-161

Schnurr D, Dondero M E (1993) Two new candidate adenovirus serotypes. Intervirol 36:79-83

Stackhouse M A, Pederson L C, Grizlle W E, Curiel D T, Gebert J, Haack K, Vickers S M, Mayo M S, Buchsbaum D J (2000). Fractionated radiation therapy in combination with adenoviral delivery of the cytosine deaminase gene and 5-fluorocytosine enhances cytotoxic and anti tumor effects in human colorectal and cholangiocarcinoma models. Gene Ther 7:1019-1026

Tsukamoto K, Ueda M, Hirata S, Osada A, Kitamura R, Takahashi T, Ichishasi M, Shimada S (2000). Gp100 mRNA is more sensitive than tyrosine mRNA for RT-PCR amplification to detect circulating melanoma cells in peripheral blood of melanoma patients. J Dermatol Sci 23:126-131

Tuting T, Steitz J, Bruck J, Gambotto A, Steinbrink K, Deleo A B, Robbins P, Knop J, Enk A H (1999). Dendritic cell based genetic immunization in mice with a recombinant adenovirus encoding murine TRP2 induces effective anti-melanoma immunity. J Gene Med 1:400-406

Weedon S J, Green N K, McNeish I A, Gilligan M G, Mautner V, Wrighton C J, Mountain A, Young L S, Kerr D J, Searle, P F (2000). Sensitation of human carcinoma cells to the prodrug CB1954 by adenovirus vector-mediated expression of E.coli nitroreductase. Int J Cancer 86:848-854

Yang S, Kittlesen D, Slingluff C L, Vervaert C E, Seigler H F, Darrow T L (2000). Dendritic cells infected with a vaccinia vactor carrying the human gp100 gene simutaneously present multiple specificities and elicit high affinity T cells reactive to multiple epitopes and restricted by HLA-A2 and -A3. J Immunol 164:4204-4211

TABLE A

| Cell line | CAR* | Ad5** | Fib11 | Fib13 | Fib16 | Fib32 | Fib35 | Fib51 |
|---|---|---|---|---|---|---|---|---|
| A549 | ++ | 10⁶ | 10⁶ | 10⁶ | 10⁶ | 10⁶ | 10⁶ | 10⁶ |
| Capan-1 | ++ | 10⁵ | 10⁵ | 10⁶ | 10⁶ | 10⁵ | 10⁶ | 10⁶ |
| MCF-7 | ND | 10⁶ | 10⁶ | ND | 10⁷ | 10⁷ | 10⁶ | 10⁶ |
| BxPC3 | ++ | 10⁶ | 10⁵ | 10⁵ | 10⁵ | 10⁴ | 10⁵ | 10⁵ |
| U-87 | −/+ | 10⁵ | 10³ | ND | 10³ | 10⁴ | 10³ | 10³ |
| SK-N-MC | −/+ | 10⁶ | 10⁶ | ND | 10⁵ | 10⁴ | 10⁴ | 10⁵ |
| MIA- | ++ | 10⁷ | 10⁷ | 10⁷ | 10⁷ | 10⁶ | 10⁷ | 10⁷ |
| PaCa-2 | | | | | | | | |
| Hs 766-T | ++ | 10⁶ | 10⁶ | 10⁶ | 10⁶ | 10⁴ | 10⁶ | 10⁶ |
| Hela | ++ | 10⁶ | ND | ND | 10⁴ | 10³ | ND | 10⁴ |
| HEP-G2 | ++ | 10⁷ | ND | ND | 10⁶ | 10⁵ | ND | 10⁵ |
| Sk-Cha-1 | ++ | 10⁵ | 10⁵ | 10⁵ | 10⁶ | 10⁴ | 10⁵ | 10⁴ |
| Mz-Cha-1 | ++ | 10⁵ | 10⁵ | 10⁵ | 10⁶ | 10⁴ | 10⁶ | 10⁶ |
| OE-19 | ++ | 10⁴ | 10⁴ | 10⁴ | 10⁵ | 10³ | 10⁴ | 10⁵ |
| OE-33 | ++ | 10⁶ | 10⁶ | 10⁶ | 10⁶ | 10⁵ | 10⁶ | 10⁷ |
| TE-1 | ++ | 10⁵ | 10⁶ | 10⁶ | 10⁶ | 10³ | 10⁶ | 10⁶ |
| TE-2 | ++ | 10⁵ | 10⁵ | 10⁵ | 10⁵ | 10³ | 10⁵ | 10⁵ |
| T-tn | ++ | 10⁵ | 10³ | 10³ | 10⁴ | 10² | 10³ | 10⁴ |

TABLE B

| Cell line | Day | Ad5 | Ad13 | Ad5Fib13 | Ad16 | Ad5Fib16 | Ad35 | Ad5Fib35 |
|---|---|---|---|---|---|---|---|---|
| U251 | 6 | +++ | − | − | + | + | + | ++ |
| U251 | 10 | ++++ | − | ++ | + | + | + | ++++ |
| HS776T | 6 | − | − | + | − | + | − | ++ |
| HS776T | 10 | ++ | − | ++++ | + | ++++ | + | ++++ |
| U87 | 6 | − | − | − | − | − | − | − |
| U87 | 10 | − | − | − | − | − | − | − |
| Capan-1 | 6 | + | − | − | ++ | +++ | ++ | +++ |
| Capan-1 | 10 | ++++ | ++++ | ++++ | ++++ | ++++ | ++++ | ++++ |
| MCF-7 | 6 | +++ | − | +++ | + | ++ | − | ++++ |
| MCF-7 | 10 | ++++ | + | ++++ | ++ | ++++ | ++++ | ++++ |
| MiaPaca | 6 | − | − | − | − | − | − | − |
| MiaPaca | 10 | − | − | − | − | − | − | − |
| BxPC3 | 6 | ++ | − | + | ++ | ++ | +++ | +++ |
| BxPC3 | 10 | ++++ | − | ++ | +++ | +++ | ++++ | ++++ |
| SK-N-MC | 6 | − | − | − | − | − | − | − |
| SK-N-MC | 10 | − | − | − | − | − | − | − |
| A549 | 6 | ++++ | +++ | ++++ | ++++ | ++++ | ++++ | ++++ |
| A549 | 10 | ++++ | ++++ | ++++ | ++++ | ++++ | ++++ | ++++ |
| PER.C6 | 6 | ++++ | ++++ | ++++ | +++ | ++++ | ++++ | ++++ |
| PER.C6 | 10 | ++++ | ++++ | ++++ | ++++ | ++++ | ++++ | ++++ |

Scoring
− 0% CPE
+ 25% CPE
++ 50% CPE
+++ 75% CPE
++++ 100% CPE

TABLE C

| CPE observed with | | | replication | | entry | | |
|---|---|---|---|---|---|---|---|
| Ad5 | AdX | Ad5FibX | Ad5 | AdX | Ad5 | Adx | situation |
| − | − | − | ? | ? | ? | ? | (1) |
| − | − | + | Y | N | N | Y | (2) |
| − | + | − | N | Y | ? | Y | (3) |
| + | − | − | Y | ? | Y | N | (4) |
| + | + | − | Y | Y | Y | Y | (5) |
| + | − | + | Y | N | Y | Y | (6) |
| − | + | + | Y | Y | N | Y | (7) |
| + | + | + | Y | Y | Y | Y | (8) |

AdX is a wild type adenovirus serotype other than adenovirus serotype 5 (Ad5).
Ad5FibX is an Ad5 wild type vector carrying the fiber of a wild type adenoviruses other than Ad5.
Scoring:
− (no CPE),
+ (CPE),
Y = Yes,
N = No

TABLE D

| Cell line | Day | Ad5 | Ad3 | Ad7 | Ad11 | Ad14 | Ad16 | Ad21 | Ad34 | Ad35 |
|---|---|---|---|---|---|---|---|---|---|---|
| U251 | 3 | + | − | − | − | − | − | − | − | − |
| U251 | 6 | +++ | − | − | + | − | − | − | − | − |
| Hs776T | 3 | − | ND | ND | − | ND | − | ND | ND | − |
| Hs776T | 6 | − | ND | ND | ++ | ND | + | ND | ND | + |
| Capan-1 | 3 | − | − | − | − | − | − | − | − | − |
| Capan-1 | 6 | + | − | − | ++++ | − | ++ | + | ++ | ++ |
| MCF-7 | 3 | − | − | − | + | − | + | − | − | − |
| MCF-7 | 6 | ++ | − | − | ++++ | − | ++ | + | ++ | − |
| BxPC3 | 3 | − | − | − | + | − | − | − | + | − |
| BxPC3 | 6 | ++ | − | − | ++++ | − | + | + | ++ | − |
| A549 | 3 | + | ++ | + | +++ | − | ++ | ++ | ++ | ++ |
| A549 | 6 | ++++ | ++++ | ++++ | ++++ | ++ | ++++ | ++++ | ++++ | ++++ |
| PER.C6 | 3 | +++ | + | +++ | ND | − | ++ | + | ++++ | ++ |
| PER.C6 | 6 | ++++ | ++ | ++++ | ++++ | + | +++ | +++ | ++++ | ++++ |

TABLE E

| Cell line | Day | Ad5 | Ad8 | Ad9 | Ad10 | Ad13 | Ad15 | Ad17 | Ad18 | Ad19 |
|---|---|---|---|---|---|---|---|---|---|---|
| U251 | 3 | + | − | − | − | − | − | − | − | − |
| U251 | 6 | +++ | − | − | − | − | − | − | − | − |
| Hs776T | 3 | − | − | ND | ND | − | ND | ND | ND | ND |
| Hs776T | 6 | − | − | ND | ND | − | ND | ND | ND | ND |
| Capan-1 | 3 | − | − | − | − | − | − | − | − | − |
| Capan-1 | 6 | + | − | − | +++ | − | − | − | − | + |
| MCF-7 | 3 | − | − | − | − | − | − | − | − | − |
| MCF-7 | 6 | ++ | − | − | + | − | − | − | − | − |
| BxPC3 | 3 | − | − | − | − | − | − | − | − | − |
| BxPC3 | 6 | ++ | − | − | + | − | + | − | − | − |
| A549 | 3 | + | ++ | + | + | − | − | + | − | − |
| A549 | 6 | ++++ | ++ | ++++ | ++++ | +++ | ++ | ++++ | ++++ | +++ |
| PER.C6 | 3 | +++ | − | − | ND | + | ++ | + | + | + |
| PER.C6 | 6 | ++++ | − | ++ | ++++ | ++++ | ++++ | +++ | ++ | + |
| Cell line | Day | Ad5 | Ad20 | Ad22 | Ad23 | Ad24 | Ad25 | Ad26 | Ad27 | Ad30 |
| U251 | 3 | + | − | − | − | − | − | − | − | − |
| U251 | 6 | +++ | − | − | − | − | − | − | − | − |
| Hs776T | 3 | − | ND | ND | ND | ND | ND | ND | ND | ND |
| Hs776T | 6 | − | ND | ND | ND | ND | ND | ND | ND | ND |
| Capan-1 | 3 | − | − | − | − | − | − | − | − | − |
| Capan-1 | 6 | + | − | − | − | − | − | − | − | − |
| MCF-7 | 3 | − | − | − | − | − | − | − | − | − |
| MCF-7 | 6 | ++ | − | − | − | − | − | − | − | − |
| BxPC3 | 3 | − | − | − | − | − | − | − | − | − |
| BxPC3 | 6 | ++ | − | − | − | − | − | − | − | − |
| A549 | 3 | + | − | − | − | − | − | − | − | − |
| A549 | 6 | ++++ | ++ | +++ | ++ | +++ | ++++ | +++ | +++ | +++ |
| PER.C6 | 3 | +++ | − | − | − | − | + | − | ++ | + |
| PER.C6 | 6 | ++++ | + | + | + | + | + | ++ | +++ | ++ |

| Cell line | Day | Ad5 | Ad32 | Ad33 | Ad36 | Ad37 | Ad38 |
|---|---|---|---|---|---|---|---|
| U251 | 3 | + | − | − | − | − | − |
| U251 | 6 | +++ | − | − | − | − | − |
| Hs776T | 3 | − | − | ND | ND | ND | ND |
| Hs776T | 6 | − | − | ND | ND | ND | ND |
| Capan-1 | 3 | − | − | − | − | − | ND |
| Capan-1 | 6 | + | − | − | − | + | ND |
| MCF-7 | 3 | − | − | − | − | − | ND |
| MCF-7 | 6 | ++ | − | − | − | − | ND |
| BxPC3 | 3 | − | − | − | − | − | ND |
| BxPC3 | 6 | ++ | − | − | − | − | ND |
| A549 | 3 | + | − | − | + | + | ND |
| A549 | 6 | ++++ | +++ | +++ | ++++ | ++++ | ND |
| PER.C6 | 3 | +++ | − | + | ++ | − | ND |
| PER.C6 | 6 | ++++ | + | + | ++++ | ++ | ND |

TABLE F

| | Ad5 | Ad5 | Ad11 | Ad11 | Ad16 | Ad16 | Ad51 | Ad51 |
|---|---|---|---|---|---|---|---|---|
| Cell line | 1^{st} | 2^{nd} | 1^{st} | 2^{nd} | 1^{st} | 2^{nd} | 1^{st} | 2^{nd} |
| A549 | ++++ | ++++ | ++++ | ND | ++++ | ++++ | ++++ | ++++ |
| BxPC3 | ++++ | +++ | ++++ | ++++ | ++++ | ++++ | ++++ | ND |
| Capan-1 | ++++ | ++ | ++++ | ND | ++++ | ND | ++++ | + |
| Hs766T | ++++ | ++ | ++++ | ++++ | ++++ | ND | ++++ | ND |
| PER.C6 | ++++ | ++++ | ++++ | ++++ | ++++ | ++++ | ++++ | ++++ |

1. A method for identifying an adenoviral replicon capable of efficiently eliminating a target cell, wherein said adenoviral replicon harbors all features essential for said adenoviral replicon to replicate in said target cell, said method comprising the steps of:
contacting said target cell with an adenoviral replicon;
determining whether said adenoviral replicon is capable of infecting said target cell more efficiently than adenovirus serotype 5; and
determining whether said target cell is efficiently eliminated by said adenoviral replicon.

2. A method for identifying an adenoviral replicon capable of efficiently eliminating a target cell, wherein said adenoviral replicon harbors all features essential for said adenoviral replicon to replicate in said target cell, said method comprising the steps of:
contacting a cell which is representative for said target cell, with an adenoviral replicon;
determining whether said adenoviral replicon is capable of infecting said representative cell more efficiently than adenovirus serotype 5; and
determining whether said representative cell is efficiently eliminated by said adenoviral replicon.

3. A method according to claim 2, wherein said representative cell is a human cell.

4. A method according to claim 2 or 3, wherein said representative cell is a tumor cell.

5. A method according to claim 4, wherein said tumor cell is a transformed brain cell, gastro-intestinal cell, pancreatic-cell, liver cell, breast tumor cell, cervix cell, lung cell or skin cell.

6. A method according to any one of claims 1-5, wherein said adenoviral replicon comprises an adenovirus.

7. A method according to claim 6, wherein said adenovirus comprises a recombinant adenovirus.

8. A method according to claim 7, wherein said recombinant adenovirus comprises a chimaeric adenovirus.

9. A method according to any one of claims 6-8, wherein said adenovirus comprises an alteration in its E1 region and/or its E3 region.

10. A method according to any one of claims 6-9, wherein said adenovirus comprises an alteration in its E1B gene.

11. A method according to claim 10, wherein said alteration results in loss of at least part of E1B-55K function.

12. A method according to any one of claims 1-11, wherein said adenoviral replicon is derived from subgroup B and/or C adenovirus, and wherein said adenoviral replicon is not wild type adenovirus serotype 5.

13. A method according to any one of claims 1-12, wherein said adenoviral replicon comprises nucleic acid comprising a fusion between nucleic acid from adenovirus serotype 5 and nucleic acid from an adenovirus serotype from subgroup B.

14. A method according to any one of claims 1-12, wherein said adenoviral replicon comprises nucleic acid comprising a fusion between at least a part of a structural protein encoding nucleic acid from a first adenovirus serotype and at least a part of a structural protein encoding nucleic acid from a second adenovirus serotype.

15. A method according to claim 14, wherein said first adenovirus serotype is adenovirus serotype 5 and wherein said second adenovirus serotype is an, adenovirus serotype from subgroup B.

16. A method according to claim 14 or 15, wherein said structural protein encoding nucleic acid encodes a fiber protein.

17. A method according to any one of claims 1-16, wherein said adenoviral replicon comprises an alteration in a gene encoding a protein involved in replication.

18. A method according to any one of claims 1-17, wherein said adenoviral replicon comprises heterologous nucleic acid.

19. A method according to claim 18, wherein said heterologous nucleic acid comprises a nucleic acid encoding a tumor-specific antigen or a functional equivalent thereof.

20. A method according to claim 19, wherein said tumor-specific antigen is selected from the group consisting of: gp100, CAMEL, PRAME, Mart-1, Mage-1, Melan-A and tyrosinase.

21. A method according to claim 18, wherein said heterologous nucleic acid comprises a nucleic acid encoding a detrimental effect inducing protein or a functional equivalent thereof.

22. A method according to claim 21, wherein said detrimental effect inducing protein is selected from the group consisting of: VP3 from chicken anemia virus, cytosine deaminase, nitroreductase, thymidine kinase and linamarase.

23. A method according to claim 18, wherein said heterologous nucleic acid comprises a nucleic acid encoding a protein capable of inducing anti-angiogenic effects, or a functional equivalent thereof.

24. A method according to claim 23, wherein said protein is a VEGF antagonist or ATF-BPTI.

25. A method according to claim 18, wherein said heterologous nucleic acid is present in an antisense form.

26. A method according to any one of claims 1-25, wherein said identification is carried out in a high-throughput format.

27. A method according to any one of claims 1-26, wherein said target cell is a human cell.

28. A method according to any one of claims 1-27, wherein said target cell is a neoplastic cell.

29. A method according to any one of claims 1-28, wherein said target cell does not efficiently express a functional p53 protein.

30. A method according to any of claims 1-29, wherein said elimination comprises lysis.

31. A method for producing an adenoviral replicon identified by a method according to any one of claims 1-30, said method for producing an adenoviral replicon comprising the steps of:
providing an adenoviral replicon identified by a method according to any one of claims 1-30;
culturing a producer-cell in a culture medium;
contacting said producer cell with said adenoviral replicon;
allowing replication of said adenoviral replicon in said producer cell; and
obtaining replicated adenoviral replicon from said producer cell and/or said culture medium.

32. A method according to claim 31, wherein said producer cell comprises a functional E1 region obtainable from adenovirus.

33. A method according to claim 31 or 32, wherein said producer cell is a PER.C6 cell or a derivative thereof.

34. A method according to claim 31 or 32, wherein said producer cell is an A549 or U87 cell or a derivative thereof.

35. A pharmaceutical composition comprising an adenoviral replicon, identified and/or obtainable by a method according to any one of claims 1-34.

36. An adenoviral replicon identified and/or obtainable by a method according to any one of claims 1-34 for use in treating a human or animal body by surgery, therapy or diagnostics.

37. Use of a pharmaceutical composition according to claim 35 for the treatment of neoplastic disease.

38. A method for treating a neoplastic condition, comprising administering a composition comprising an adenoviral replicon capable of eliminating a neoplastic cell to an individual having a neoplasm.

39. A method according to claim 38, wherein said neoplasm comprises a tumor.
